(19) United States
(12) Patent Application Publication
(10) Pub. No.: US 2016/0278643 A1
(43) Pub. Date: Sep. 29, 2016

(54) BIOPHOTONIC MEASUREMENT APPARATUS AND BIOPHOTONIC MEASUREMENT METHOD USING SAME

(71) Applicant: HITACHI, LTD., Tokyo (JP)

(72) Inventors: Masashi KIGUCHI, Tokyo (JP); Tsukasa FUNANE, Tokyo (JP)

(21) Appl. No.: 15/032,772

(22) PCT Filed: Dec. 18, 2013
(86) PCT No.: PCT/JP2013/083817

§ 371 Date: Apr. 28, 2016

(51) Int. Cl. A61B 5/026; A61B 5/00

(52) U.S. Cl. CPC A61B 5/0261; A61B 5/7425; A61B 5/746; A61B 5/7203; A61B 5/4064; A61B 2562/0238

(57) ABSTRACT

Signals derived from the brain or cerebral cortex are extracted by separating and removing influence of the skin blood flow included in NIRS signals. Provided is a biophotonic measurement apparatus to separate signals simultaneously measured with a plurality of irradiator-detector distances (SD distances) into brain blood flow-derived signals and skin blood flow-derived signals using SD distance dependency of signal amplitudes.

### TECHNICAL FIELD

The present invention relates to a technique to separate and remove influence of surface layer components such as skin blood flow components mixed in signal components using a biophotonic measurement apparatus using visible light or near infrared light.

### BACKGROUND ART

It is reported that optical detection signals and biological signals (hereinafter referred to as NIRS signals) obtained from non-invasive optical brain function imaging using NIRS including optical topography may be affected by variations in skin blood flow in the scalp since irradiation with light is performed on/over the scalp. In consideration of such influence of skin blood flow, methods to extract and remove components thereof are studied. Most of such methods acquire signal components from portions with different depths by method using a plurality of irradiator-detector (source-detector) distances (hereinafter referred to as SD distance) and intend to remove, using the signal components, skin blood flow-derived signals which may affect measurement signals of a shallow layer part. A method of measuring with a plurality of SD distances is hereinafter referred to as a multi-SD method.

For example, there is a method where absorption coefficients in the scalp and brain (grey matter) are determined by system of equations using optical path lengths in the scalp and brain (grey matter) with each of a short SD distance and long SD distance (e.g. refer to NPL 2). In this method, a configuration of the head is assumed as a two-layered model and a partial mean optical path length in each of the layers is further required to be assumed; however, assuming an optical path length of a subject is difficult.

Also, a subtraction method using an adaptive filter has been proposed. In this method, by subtracting, from a measurement signal with a long SD distance (hereinafter referred to as long SD signal), a value obtained by multiplying a measurement signal with a short SD distance (hereinafter referred to as short SD signal) by an appropriate coefficient, a skin blood flow-derived signal is removed (e.g. refer to NPL 3). Moreover, as the subtraction method using linear regression, a method is proposed where brain activity signals are obtained by subtracting, from a long SD signal, a fitting signal where short SD signals are fitted to linear regression of long SD signals (e.g. refer to NPL 4).

As techniques related to the above, the methods below are disclosed.

PTL 2 discloses a method to dispose a plurality of pairs of irradiator-detector such that midpoints thereof are equivalent with each other, to perform measurement thereby, and to remove unnecessary information by arithmetic processing with an object of providing an optical measurement apparatus capable of removing unnecessary information derived from skin blood flow or the like using an optical emitting/receiving unit having a plurality of light emitting probes and a plurality of light receiving probes. Also, PTL 3 discloses a method to obtain a result characterizing a state mainly in the brain tissue itself free from influence derived from overlapping adjacent tissues or the like by employing a device configuration where two detectors are used for one light source and appropriately discriminating information obtained from the two detectors. Furthermore, PTLs 4, 5, and 6 disclose methods to calculate variations in absorbance and to perform calculation such as subtraction with a long SD signal and short SD signal. These methods, however, have problems as described below.

Firstly, there is a problem that it is difficult to determine various coefficients in calculations such as subtraction among measurement signals of respective SD distances. In such calculation, various coefficients affect the result and thus it is required to set appropriate values. Moreover, when short SD signals are acquired, an SD distance of 10 mm is often provided and thereby signal components not affected by brain blood flow but by variations in absorbance only in the skin are acquired. Therefore, an amplitude ratio of brain blood flow-derived components and skin blood flow-derived components is unknown and thus determining an appropriate coefficient for the calculation is difficult. In order to appropriately correct long SD measurement signals including contributions from the skin and brain, it is required to know contribution ratios of each of the skin and brain and their optical path length ratio.

Furthermore, fitting short SD signals to long SD signals may disadvantageously remove brain blood flow-derived signals from the long SD signals when skin blood flow-derived signals and brain blood flow-derived signals are not independent, that is, the skin blood flow-derived signals and brain blood flow-derived signals are correlated.

As a means to solve the above problems, PTL 7 discloses a method to separate, for each independent component calculated using independent component analysis, signals measured with a plurality of SD distances into brain blood flow-derived signal components and skin blood flow-derived signal components using SD distance dependency and to restructure the brain blood flow-derived signals and skin blood flow-derived signals for each of the SD distances.

However, since this method uses the independent component analysis, measurement signals are required to be time series signals of a certain period of time. Thus, processing to separate skin blood flow-derived signals and brain blood flow-derived signals is performed after measurement and no result can be obtained during measurement.

### CITATION LIST

### Patent Literature

PTL 1: JP 09-019408 A

PTL 2: JP 2008-64675 A

PTL 3: Japanese National Publication of International Patent Application No. 2002-527134

PTL 4: U.S. Pat. No. 7,072,701 B2

PTL 5: U.S. Pat. No. 5,349,961

PTL 6: U.S. Pat. No. 5,902,235

PTL 7: WO 2012005303 A1

PTL 8: JP 2001-178708 A

### Non-Patent Literature

### SUMMARY OF INVENTION

### Technical Problem

An object of the present invention is to separate and extract brain blood flow-derived signals and skin blood flow-derived signals included in NIRS signals during measurement.

### Solution to Problem

In the present invention, in particular, measured values of variations in hemoglobin concentration-length measured at a plurality of SD distances at timings that can be deemed as simultaneous are separated into skin blood flow-derived signals and brain blood flow-derived signals in real-time using SD distance dependency.

### Advantageous Effects of Invention

The present invention allows for performing measurement while brain blood flow-derived signals and skin blood flow-derived signals are separated and extracted in real time during measurement. This allows for interrupting and redoing the measurement when the brain blood flow-derived signals cannot be measured with a sufficient accuracy, thereby enabling efficient and sound data acquisition.

### BRIEF DESCRIPTION OF DRAWINGS

FIG. 1 is a diagram illustrating a configuration of an apparatus of the present invention.

FIG. 2 is a diagram illustrating an exemplary measurement cross-sectional view of a multi-SD method.

FIGS. 3(a) and 3(b) are diagrams illustrating relationships between the SD distance and partial mean optical path lengths in the scalp and grey matter, respectively.

FIG. 4 is a diagram illustrating an SD dependency model of variations in hemoglobin concentration-length in the scalp and grey matter.

FIG. 5 is a diagram illustrating exemplary arrangement of probes on the human head.

FIG. 6 is a diagram illustrating exemplary latticed arrangement of probes and arrangement of measurement points of the related art.

FIGS. 7(a) and 7(b) are diagrams illustrating exemplary arrangement of probes and arrangement of measurement points of doubled density, respectively.

FIG. 8 is a diagram illustrating a flowchart of measurement.

FIG. 9 is a diagram illustrating a test configuration using an optical brain function measurement apparatus of a whole head measurement type.

FIG. 10 is a diagram illustrating exemplary display of signals separated and extracted during measurement.

FIG. 11 is a diagram illustrating exemplary 2D-display of signals separated and extracted during measurement.

FIG. 12 is a diagram illustrating exemplary 2D-display of signals separated and extracted during measurement.

### DESCRIPTION OF EMBODIMENTS

Embodiments of the present invention will be described below with reference to the drawings.

### Example 1

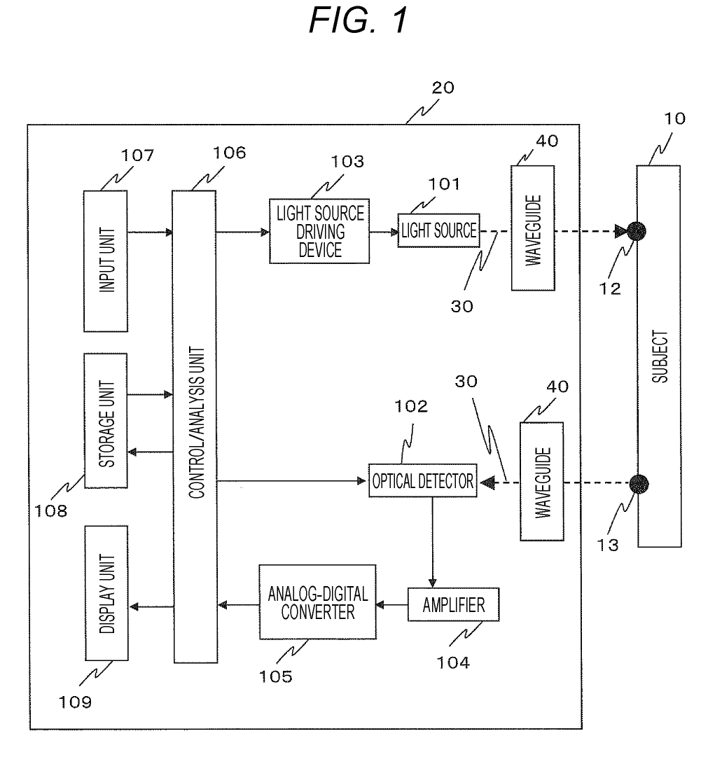

An exemplary configuration of an apparatus of the present invention is illustrated in FIG. 1. With a biophotonic measurement apparatus capable of emitting light to a living body and detecting light output from the living body after diffusion, absorption, and propagation therein, light 30 irradiated from one or more light sources 101 included in an apparatus main body 20 enters a subject 10 via a waveguide 40. The light 30 enters inside the subject 10 from an irradiation point 12, is transmitted and propagated within the subject 10, and then detected by one or more optical detectors 102 via a waveguide 40 from a detection point 13 located away from the irradiation point 12. An SD distance is defined by, as described above, a distance between the irradiation point 12 and detection point 13.

Here, the one or more light sources 101 are only required to be semiconductor lasers (LDs), light-emitting diodes (LEDs), or the like and the one or more optical detectors are only required to be avalanche photodiodes (APDs), photodiodes (PDs), photomultiplier tubes (PMTS), or the like. Also, the waveguide 40 is only required to be an optical fiber, glass, light guide, or the like.

The light source 101 is driven by a light source driving device 103. A gain of the one or more optical detectors 102 is controlled by a control/analysis unit 106. The control/analysis unit 106 also controls the light source driving device 103 and receives input of conditions or the like from an input unit 107.

Electrical signals photoelectrically converted by the optical detector 102 are amplified by an amplifier 104, subjected to analog-digital conversion by an analog-digital converter 105, transmitted to the control/analysis unit 106, and processed thereat.

The control/analysis unit 106 has a control unit to control the light source driving device and/or optical detector and an analysis unit to analyze signals obtained from the optical detector and executes analysis based on signals detected by the optical detector 102. Specifically, with digital signals obtained by conversion by the analog-digital converter 105, variations in oxy-hemoglobin (oxy-Hb) concentration-length and/or deoxy-hemoglobin (deoxy-Hb) concentration-length are calculated from variations in detected amount of light or variations in absorbance based on, for example, a method described in NPL 1. Here, the variation in concentration-length refers to an amount of variation in a product of concentration and optical path length.

Descriptions herein assume that the control/analysis unit 106 performs all of driving the light source 101, gain control of the optical detector 102, and signal processing from the analog-digital converter 105; however, the same functions can be implemented by providing separate control units and further providing a means to integrate these control units.

Furthermore, detection signals of received light amount and signals of variations in oxy-hemoglobin or deoxy-hemoglobin concentration-length calculated using the detection signals are stored in a storage unit 108. Measurement results can be displayed on a display unit 109 based on an analysis result and/or stored data.

Although a source 50 and detector 60 are not illustrated in FIG. 1, the source 50 includes, for example, the waveguide 40 on the light source 101 side and mounted to be in contact or in a state close to be in contact with the subject 10. The detector 60 includes, for example, the waveguide 40 on the optical detector 102 side and mounted to be in contact or in a state close to be in contact with the subject 10. Here, the respective sources 50 and detectors 60 are arranged on/over the subject 10 such that light received by the respective detectors is propagated in both of the grey matter and scalp.

Next, a method to separate and extract the brain blood flow-derived signals and skin blood flow-derived signals will be described. Hereinafter, of variations in hemoglobin concentration-length obtained from NIRS measurement, brain blood flow-derived will be described only for variations in oxy-hemoglobin concentration-length with a large amplitude; however, variations in deoxy-hemoglobin (deoxy-Hb) concentration-length or variations in total hemoglobin (oxy-Hb+deoxy-Hb) concentration-length may be used. When it is simply noted as variations in hemoglobin concentration-length, the term is used in a generic sense and may include any of the above.

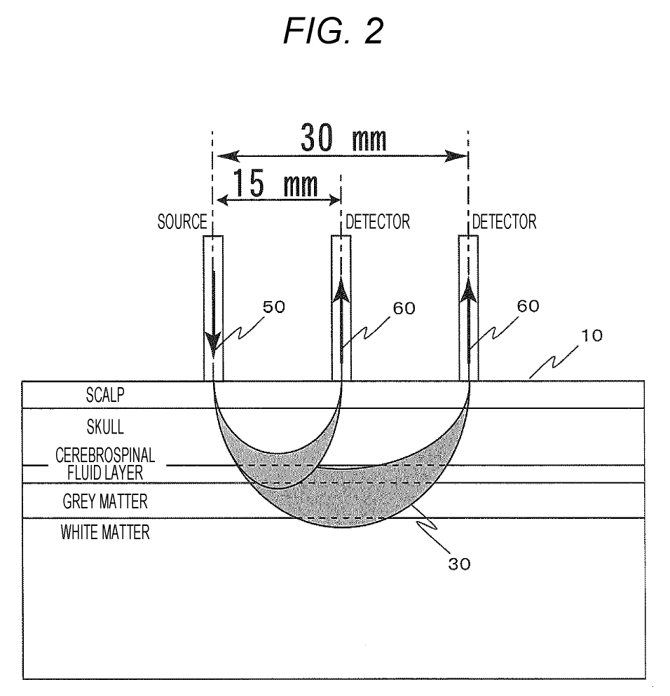

An exemplary measurement cross-sectional view of a multi-SD method is illustrated in FIG. 2. The light 30 irradiated from the source 50 is incident on the scalp and is propagated in all directions within tissues. When the detectors 60 are arranged at SD distances of 15 mm and 30 mm as illustrated in FIG. 2, the light 30 received by the detector 60 at the SD distance of 15 mm is transmitted by a shallow part on the average as compared to the light 30 received by the detector 60 at the SD distance of 30 mm. Note that the SD distance is set to be larger than approximately 10 mm.

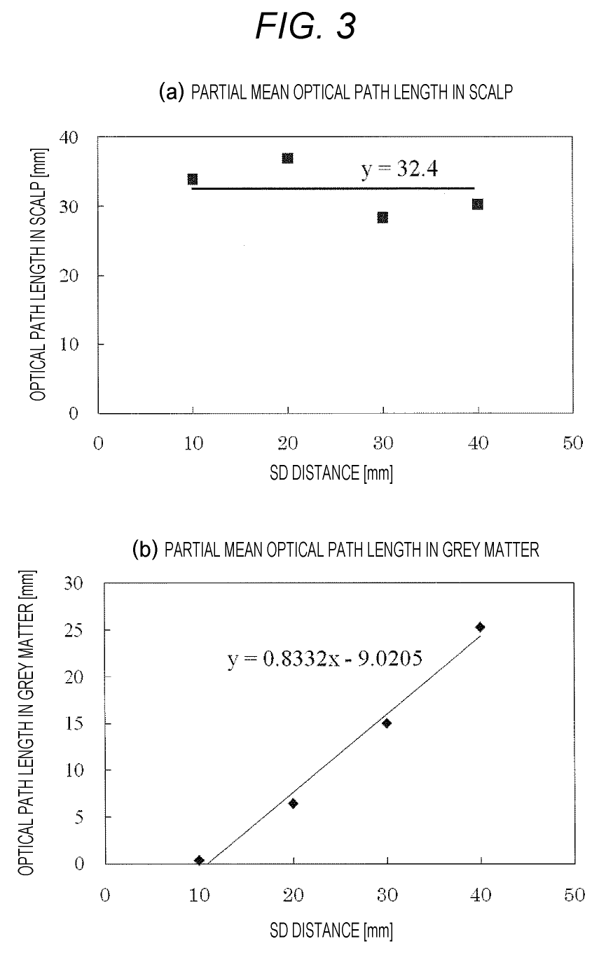

FIGS. 3(a) and 3(b) are diagrams illustrating relationships between the SD distance and partial mean optical path lengths in the scalp and grey matter obtained by the Monte Carlo simulation. FIG. 3(a) illustrates the relationship with the scalp and FIG. 3(b) illustrates the relationship with the grey matter. The horizontal axes represent the SD distance [mm] and the vertical axes represent the partial mean optical path length [mm] in the scalp and grey matter. The partial optical path length in the scalp shows no SD distance dependency while that in the grey matter shows linear SD distance dependency. The reason why the partial mean optical path lengths in the scalp are dispersed is because the number of photons calculated in the simulation is small and thus results have not converged. Since NIRS signal intensity is proportional to the partial optical path length in a portion having variations in blood flow (cf. NPL 1) (uniform variations in the blood flow are assumed in the partial optical path), it is understood from FIGS. 3(a) and 3(b) that, with a larger SD distance, brain blood flow-derived components in the variation signals of oxy-hemoglobin concentration-length become lager while skin blood flow-derived components do not vary. In the present invention, an amount of variation in the signal amplitude in relation to the SD distance, namely, a gradient (inclination) is noted.

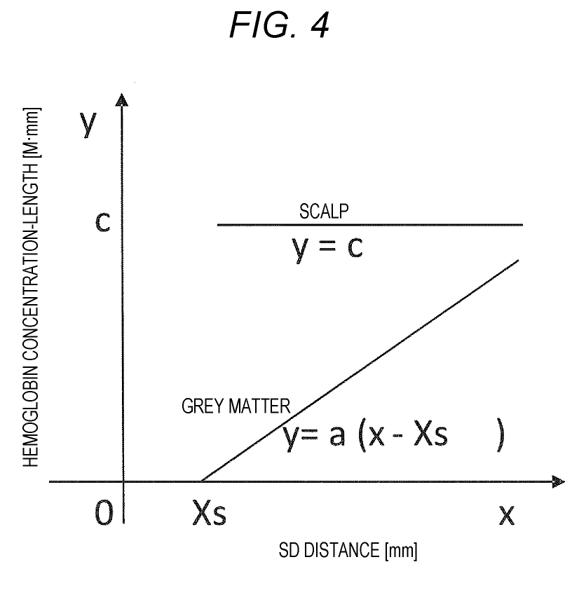

Hereinafter, a method to separate and extract brain blood flow-derived signals and skin blood flow-derived signals will be described. FIG. 4 illustrates skin blood flow-derived signals and brain blood flow-derived signals modeled from the simulation results illustrated in FIGS. 3(a) and 3(b). The horizontal axis represents the SD distance [mm] and the vertical axis represents variations in the hemoglobin concentration-length. These models can be expressed as formula 1 and formula 2.

[Formula 1]

y=c (1)

[Formula 2]

y=a(x−xs0) (2)

A measurement signal of a variation in the hemoglobin concentration-length can be expressed as formula 3 as the sum of the two.

[Formula 3]

y=a(x−xs0)+c (3)

Incidentally, here the letter y represents variations in hemoglobin concentration-length, x represents the SD distance, xs0 represents an x-intercept, a represents the inclination, and c represents a variation in the hemoglobin concentration-length derived from the skin blood flow. Since a plurality of variation values of the hemoglobin concentration-length with different SD distances is observed at timings deemed as simultaneous, the value xs0 is given. Performing linear regression according to formula 3 using these signals gives values at time t, namely a(t) and c(t). Assigning the values to formula 1 and formula 2 gives a value of skin blood flow-derived signal amplitude at that time and a value of brain blood flow-derived signal amplitude with an arbitrary SD. Therefore, repeating the above each time an observation value is obtained results in skin blood flow-derived signals in time series and brain blood flow-derived signals in time series. Here, the value xs0 corresponds to the shortest SD distance where light can reach the brain and can be deemed as constant irrespective of time. In this manner, in the present invention, the skin blood flow-derived signal and/or brain blood flow-derived signal associated with the measurement time is calculated from a plurality of measurement signals measured in association as signals at certain measurement time using SD distance dependency of the measurement signals. The value of xs0 may be obtained by the Monte Carlo simulation using a head structure of the subject. Alternatively, a value obtained from the Monte Carlo simulation using a standard head structure or a value empirically obtained may be preset. Further alternatively, an arbitrary value may be set by inputting the value externally. Still alternatively, the value xs0 may be actually measured and thereby set by blocking skin blood flow by applying pressure to the skin and measuring brain activities with a plurality of SD distances.

For example, a case of performing two types of measurement with SD distances of 15 mm and 30 mm will be described. When, at time t, a variation in the oxy-hemoglobin concentration-length measured with an SD distance of 30 mm is y30 and a variation in the oxy-hemoglobin concentration-length measured with an SD distance of 15 mm is y15, an inclination at time t, a(t), is given by formula 4.

a(t)=(y30(t)−y15(t))/(30−15) [Formula 4]

In the variation in the oxy-hemoglobin concentration-length measured with the SD distance of 30 mm, a value of brain blood flow-derived signal amplitude at time (t) is a(t)*(30−xs0) and a value of skin blood flow-derived signal amplitude at time (t) is c(t). Assigning these values to the formula 5 and formula 6 allows for obtaining a brain contribution ratio and skin contribution ratio at time t. Also with variations in the deoxy-hemoglobin concentration-length, a value of brain blood flow-derived signal amplitude, a value of skin blood flow-derived signal amplitude, brain contribution ratio, and skin contribution ratio at time t can be obtained in a similar manner.

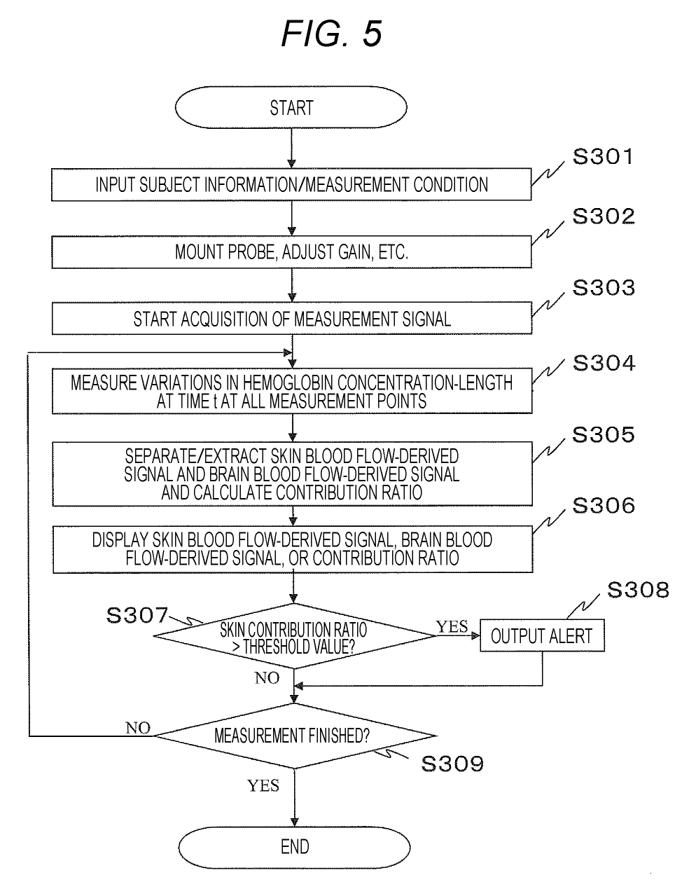

A flowchart of measurement is illustrated in FIG. 5. When measurement is started, subject information or various measurement conditions is/are set (S301). The various measurement conditions include, for example, measurement time, the number of measurement, and a threshold value of skin contribution ratio where an alert should to be issued. Furthermore, preparations for measurement such as mounting probes or adjusting the gain are provided (S302). Incidentally, the steps S301 and S302 may be vice versa. Thereafter, acquisition of measurement signals is started (S303) and signals of variations in the hemoglobin concentration-length are measured at measurement time t at all measurement points (S304). Successively, separation and extraction of skin blood flow-derived signals and brain blood flow-derived signals and calculation of the skin contribution ratio and brain contribution ratio is performed using these signals (S305). The signals and ratios are then displayed (S306). The skin contribution ratio is compared to a preset threshold value (S307). When the ratio exceeds the threshold value, an alert is output (S308). Whether to end the measurement is determined according to whether preset measurement time or the number of measurement has been reached or a user has ordered to halt the measurement (S309). When the measurement is not ended, the steps from S304 to S309 are repeated.

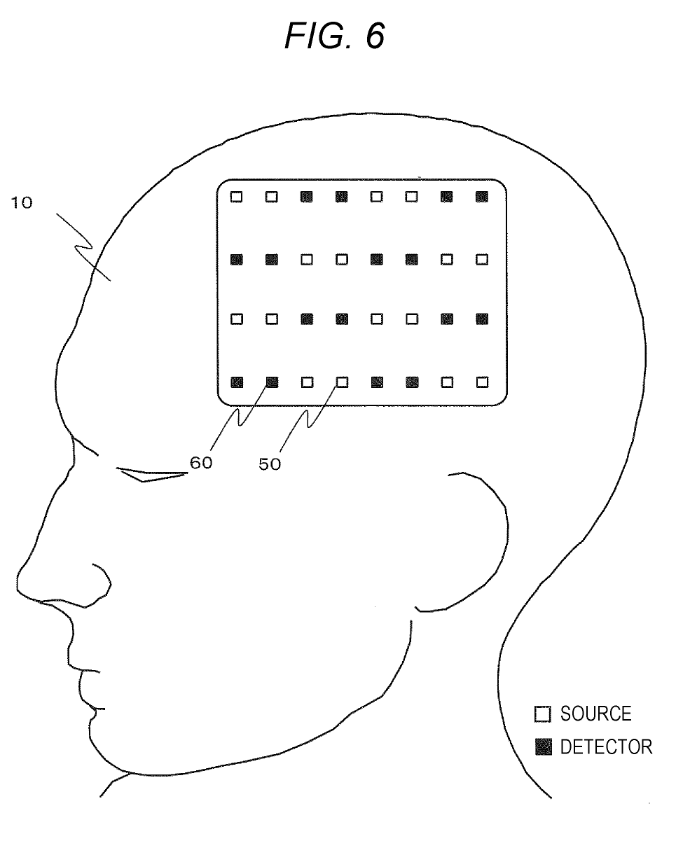

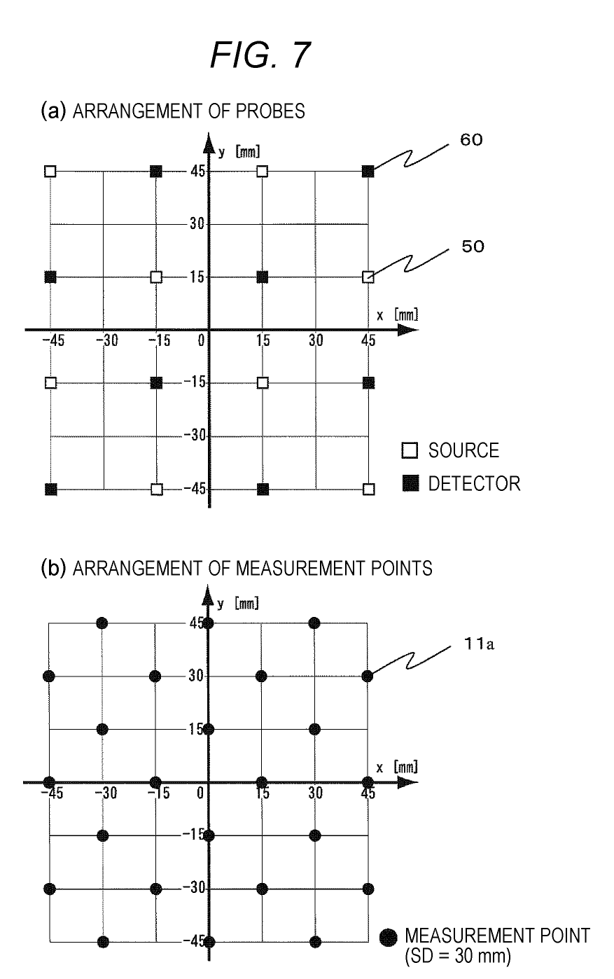

Next, a case where the above method is applied to measurement with actual arrangement of probes will be described. Exemplary arrangement of probes on the human head is illustrated in FIG. 6. The probes can be mounted on the whole head including a forehead part, side part, top part, and rear part of the head. Latticed arrangement of probes and arrangement of measurement points of the related art (e.g. refer to NPL 1) are illustrated in FIGS. 7(a) and 7(b), respectively. In this arrangement, normally an interval between the source 50 and detector 60 is approximately 30 mm and a substantial midpoint thereof is a measurement point 11a. Symbols of “□”, “▪”, and “” represent the source, detector, and measurement point, respectively. In this arrangement, an SD distance is 30 mm at all measurement points 11a. Measurement with combinations of an SD distance of 60 mm is also possible; however, this is not practical since a signal-to-noise ratio (SNR) is small.

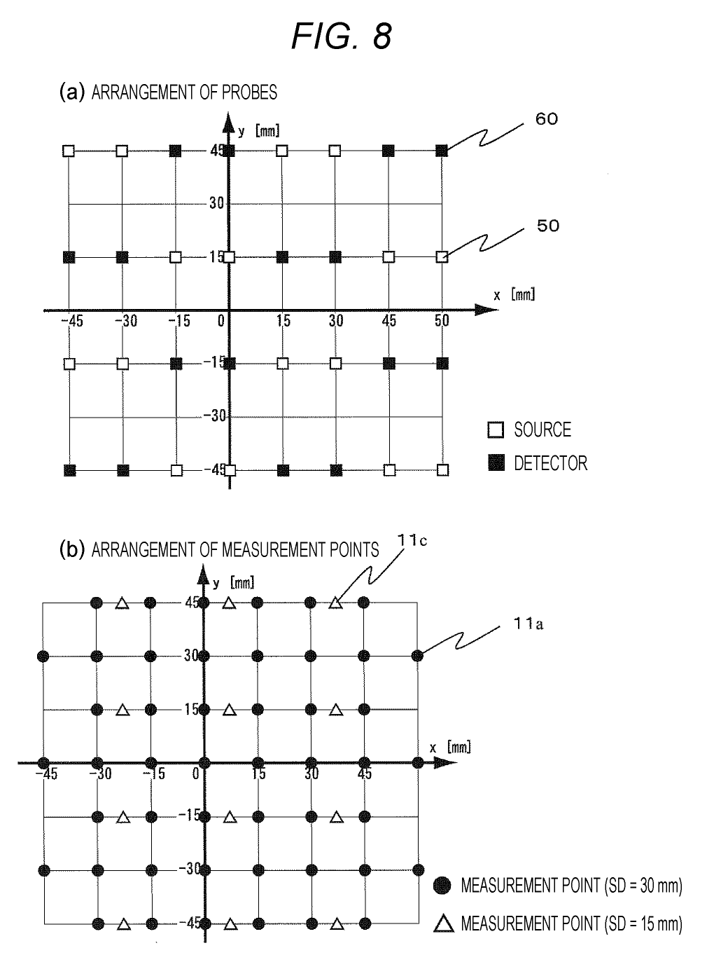

Arrangement of probes and arrangement of measurement points of doubled density are illustrated in FIGS. 8(a) and 8(b), respectively. The arrangement of probes is disclosed in PTL 8. In this arrangement, the latticed arrangement of probes in FIG. 7(a) is shifted by 15 mm along the x axis and overlaid thereon. Symbols of “□”, “▪”, “”, and “Δ” represent the source 50, detector 60, measurement point 11a with the SD distance of 30 mm, and measurement point 11a of the SD distance of 15 mm, respectively.

Here, to extract skin blood flow-derived signals, measurement signals at measurement points with a plurality of SD distances are used. When measurement signals with the same SD distance are used for mapping by interpolation, for example an SD distance is approximately 15 to 20 mm, obtained is a map with large contribution of signals derived from shallow parts including the skin.

Here, depending on an SD distance, there are cases where resolution becomes low due to a small number of measurement points when imaging is performed only with signals with the same SD distance. In the example of FIG. 8(b), the measurement points with the SD distance of 15 mm is smaller in the number as compared to the measurement points with the SD distance of 30 mm and thus distribution density thereof is lower. Even such measurement signals with the SD distance of which distribution density is low is effective for extracting signals (brain blood flow-derived signals/skin blood flow-derived signals) to be separated from the signals at the measurement points with the SD distance of 30 mm. Thus, even the number of measurement points is small, valid measurement data can be obtained.

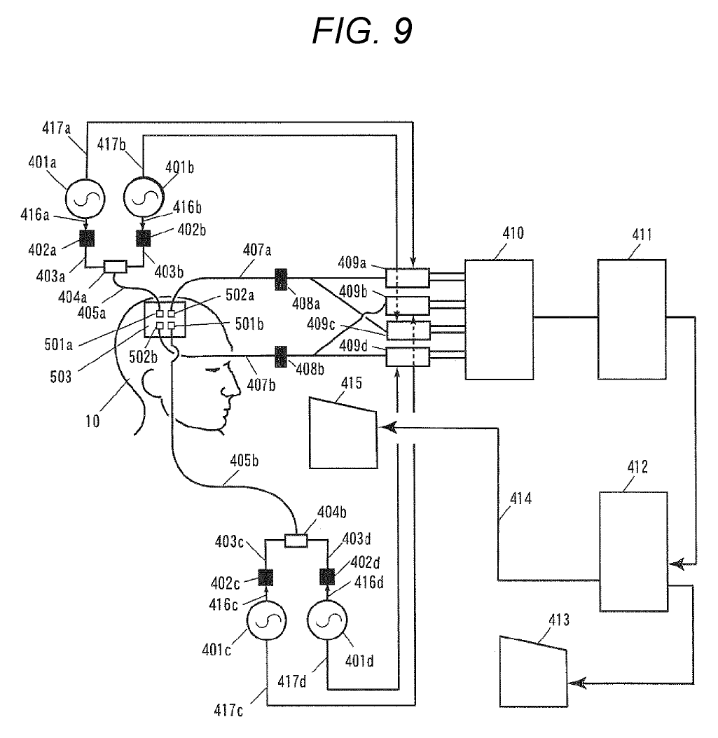

A test configuration diagram using an optical brain function measurement apparatus 90 of a whole head measurement type is illustrated in FIG. 9. A local volume of brain blood (variations in oxy-hemoglobin/deoxy-hemoglobin/total hemoglobin concentration-length) can be obtained by irradiating the head of the subject with light of wavelengths belonging to visible to infra-red regions with the optical brain function measurement apparatus 90 and detecting and measuring, by the same optical detector, light of signals of the plurality of wavelengths passed inside the subject. It is also possible to provide appropriate stimulation/order to the subject 10 during a measurement period by a stimulation/order presenting device 415. The stimulation/order presenting device 415 is controlled by a computer 412 with control signals 414.

Provided are a plurality of light sources 402a to 402d of different wavelengths (if two types of wavelengths are used, for example the light sources 402a and 402c are 695 nm and the light sources 402b and 402d are 830 nm), modulators or oscillators 401a and 401b (401c and 401d) to modulate intensity of light from the plurality of light sources 402a and 402b (402c and 402d) via driving signal lines 416a and 416b (416c and 416d) at frequencies different from one another, a plurality of light irradiation means to irradiate the scalp of the subject 10, via a light emitting optical fiber 405a (405b) with light from a coupler 404a (404b) coupling rays of the intensity-modulated light via the respective optical fibers 403a and 403b (403c and 403d), and a plurality of light receiving means formed by detectors 408a and 408b provided to each of light receiving optical fibers 407a and 407b such that tips thereof are positioned near light irradiation points of the plurality of light irradiation means at preset distances from the light irradiation points (e.g. 15 mm and 30 mm). The light passed through the living body is collected by the light receiving optical fibers 407a and 407b, then subjected to photoelectric conversion by the detectors 408a and 408b, and amplified. Here, tips of the light emitting optical fibers 405a and 405b and light receiving optical fibers 407a and 407b have light emitting probes 501a and 501b and light receiving probes 502a and 502b, respectively, for retaining the optical fibers and appropriately mounting on the subject 10. Moreover, a probe holder 503 is fixed to the subject 10 in order to retain the plurality of probes.

The light receiving means detects light reflected and/or transmitted by the inside of the subject 10 and converts the light into electrical signals. As the detector 408, a photoelectric conversion element represented by a photomultiplier tube or photodiode is used. A case of using two types of wavelengths is described in FIG. 9; however, three or more types of wavelengths may also be used. Note that two light irradiation means and two light receiving means are arranged in FIG. 8(a) for simplicity; however in the present example, a plurality of light receiving means not illustrated is included since multi-SD arrangement is needed.

The electric signals representing intensity of light passed through the living body and subjected to photoelectric conversion by the detectors 408a and 408b are respectively input to lock-in amplifiers 409a to 409d. The lock-in amplifiers 409a to 409d are also input with reference signals 417a to 417d from the oscillators [modulators] 401a and 401b (401c and 401d). For example, the lock-in amplifiers 409a and 409b output separated light of 695 nm from the light sources 402a and 402c and extracted by lock-in processing and the lock-in amplifiers 409c and 409d output separated light of 830 nm from the light sources 402b and 402d. Here, in FIG. 9 two measurement points of one between the light emitting probe 501a and light receiving probe 502a and one between the light emitting probe 501b and light receiving probe 502b are assumed for simplicity. With a similar configuration, two measurement points of one between the light emitting probe 501a and light receiving probe 502b and one between the light emitting probe 501b and light receiving probe 502a can be assumed.

Intensity signals, of the passed light of separated respective wavelengths, which are output from the lock-in amplifiers 409a to 409d are subjected to analog-digital conversion by an analog-digital converter 410 and then transmitted to a computer for measurement control 411. The computer for measurement control 411 calculates variations in the oxy-hemoglobin concentration, deoxy-hemoglobin concentration-length, and total hemoglobin concentration-length from detection signals at the respective detection points using the intensity signals of the passed light by a well-known procedure described in NPL 1 or the like and stores in a storage device as time series information at the plurality of measurement points. Incidentally, an example of performing the lock-in processing and then performing the analog-digital conversion has been described here; however, it is also possible to perform the analog-digital conversion of the signals from the detectors and then digitally performing the lock-in processing. Furthermore, the example of separating a plurality of rays of light by a modulation method has been described; however, without limiting thereto, it is also possible to use, for example a time division method where a plurality of rays of light is discriminated by shifting, in terms of time, timings of irradiating with the plurality of rays of light. In this case, if the shifted time of timings is set sufficiently short to allow approximation thereof as long as a value of hemoglobin concentration-length does not vary, measurement values can be deemed as simultaneous measurement values.

Even when timings of irradiation or detection are shifted in order to avoid saturation of the detectors, variation values of the hemoglobin concentration-length that can be deemed as simultaneous values can be measured in a similar manner.

The computer 412 includes an input unit, analysis unit, storage unit, and an extraction unit and analyzes, in the analysis unit, a result calculated by the computer for measurement control 411. The input unit is input externally with settings such as analysis conditions. Note that when the computer 412 has a display function, the display unit 413 may not be included therein. The analysis result from the analysis unit is stored in the storage unit. The extraction unit extracts information of local brain hemodynamics of the subject 10 from signals analyzed by the analysis unit. The information of local brain hemodynamics of the subject 10 extracted by the extraction unit is displayed on a display unit 413. The computer for measurement control 411 and computer 412 are separately illustrated in FIG. 9; however, they may be included in one computer.

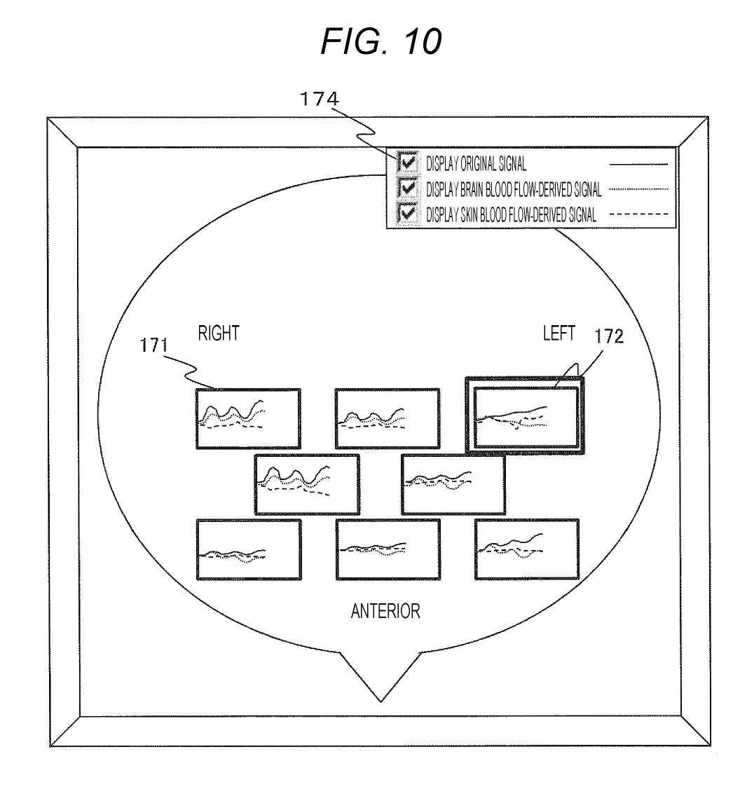

Exemplary display is illustrated in FIG. 10 where measurement is performed while variation signals of the hemoglobin concentration-length are being separated and extracted into brain blood flow-derived signals and skin-derived signals by employing the method of the present invention. Measurement signals 171 are displayed while arranged at a corresponding measurement position. As values of variation signals of the hemoglobin concentration-length are acquired, separation and extraction into the brain blood flow-derived signals and skin-derived signals are performed in real time and display of the respective waveforms is updated. To measurement signals where the skin contribution ratio exceeds the preset threshold value, an alert 172 is issued. A checkbox 173 for selecting display method of the original signals, brain blood flow-derived signals, and skin blood flow-derived signals allows for selecting measurement signals to display and implementing a display method that meets a purpose. Furthermore, although not illustrated in FIG. 10, variations in the oxy-hemoglobin concentration-length, variations in the deoxy-hemoglobin concentration-length, and variations in the total hemoglobin concentration-length may be made selectable. In this manner, issuing the alert in real time when the skin contribution ratio is excessive allows for prompt reaction such as redoing the measurement. The example of alert displayed on a screen has been illustrated here; however, an alert by sound, letters, or the like may be used. Also, a reference of alert display is the skin contribution ratio; however, the brain contribution ratio may be used.

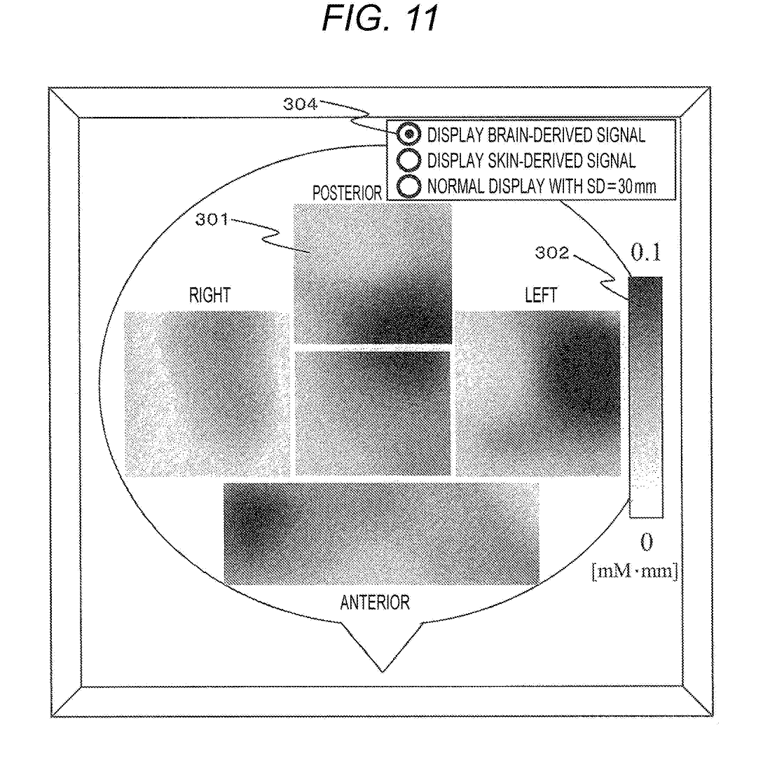

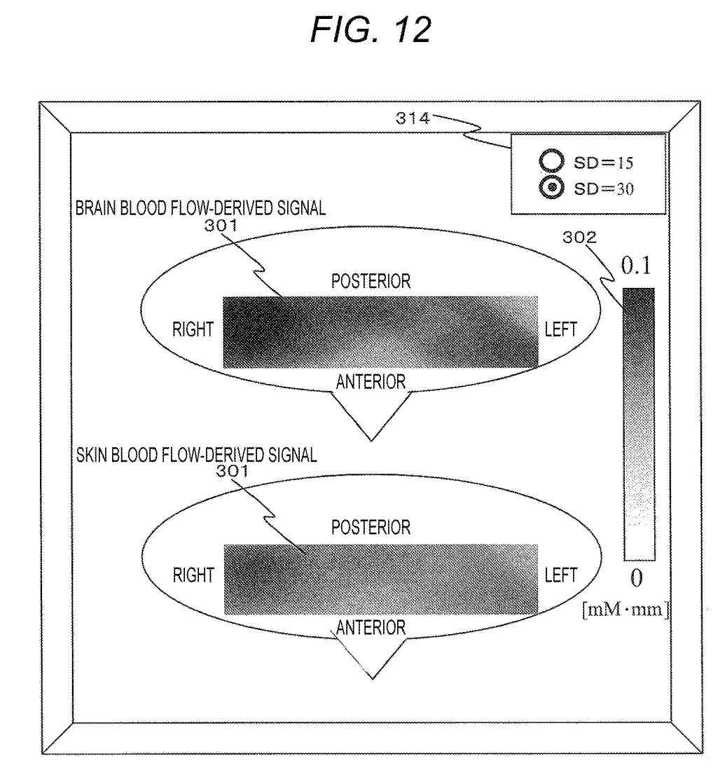

Exemplary display is illustrated in FIG. 11 where the plurality of sources 50 and detectors 60 is two-dimensionally arranged and thereby the brain blood flow-derived signals and skin blood flow-derived signals are measured with imaging. This is an exemplary display during measurement by the optical brain function measurement apparatus of the whole brain measurement type. A map of variations in the oxy-hemoglobin (oxy-Hb) concentration-length 301 of each of a forehead part, top part, left and right side parts, and rear part of the head is displayed. An amplitude value is represented by shades shown in a grey scale bar 302. Moreover, the brain-derived signals, skin-derived signals, or a normal display with the SD distance of 30 mm can be selected by a radio button 304. Furthermore, an example of comparative display of two-dimensional data of the both components is illustrated in FIG. 12. The upper diagram illustrates brain blood flow-derived signals and the lower diagram illustrates skin blood flow-derived signals. The SD distance can be switched by a radio button 314. This allows for confirming a distribution state of the brain blood flow-derived signals and skin blood flow-derived signals at a glance.

The skin contribution ratio and brain contribution ratio will be described.

[Formula 5]

Brain contribution ratio=100×value of brain blood flow-derived component signal amplitude/(value of brain blood flow-derived signal amplitude+value of skin blood flow-derived signal amplitude)[%] (13)

[Formula 6]

Skin contribution ratio=100×value of skin blood flow-derived component signal amplitude/(value of brain blood flow-derived signal amplitude+value of skin blood flow-derived signal amplitude)[%] (14)

The separated signals are displayed in FIGS. 10 to 12; however, the brain contribution ratio or skin contribution ratio may be displayed in a similar manner. This allows for grasping a distribution state of contribution ratios of the skin blood flow-derived signal components and brain blood flow-derived signal components for each portion of the subject, thereby allowing for, for example utilization for optimum selection of a task.

### INDUSTRIAL APPLICABILITY

The present invention allows for separating and extracting, from measurement signals, brain blood flow-derived components and skin blood flow-derived components in real-time depending on an object with a human head optical measurement apparatus using visible light or near infrared light, thereby enhancing accuracy and reproducibility of human brain function measurement.

### REFERENCE SIGNS LIST

10 subject
11 measurement point
11a measurement point (SD=30 mm)
11c measurement point (SD=15 mm)
12 irradiation point
13 detection point
20 apparatus main body
30 light
50 source
60 detector
90 optical brain function measurement apparatus
101 light source
102 optical detector
103 light source driving device
104 amplifier
105 analog-digital converter
106 control/analysis unit
107 input unit
108 storage unit
109 display unit
171 measurement signal
172 alert
173 checkbox for selecting display method of original signals, brain blood flow-derived signals, and skin blood flow-derived signals
301 map of variations in oxy-hemoglobin (oxy-Hb) concentration-length
302 grey scale bar
304 radio button
314 radio button
401 oscillator (modulator)
402 light source
403 optical fiber
404 coupler
405 light emitting optical fiber
407 light receiving optical fiber
408 detector (including amplifier)
409 lock-in amplifier
410 analog-digital (A/D) converter
411 computer for measurement control
412 computer
413 display unit
414 control signal
415 stimulation/order presenting device
416 light source driving signal
417 reference signal from oscillator (modulator)
501 light emitting probe
502 light receiving probe
503 probe holder

1. A biophotonic measurement apparatus, comprising:
one or more light irradiation means configured to irradiate a subject with light;
one or more light detection means configured to detect, at a detection point on/over the subject, the light emitted to an irradiation point on/over the subject from the one or more light irradiation means and propagated within the subject;
a control unit configured to control the one or more light irradiation means and the one or more light detection means;
an analysis unit configured to analyze a signal obtained by the one or more light detection means; and
a display unit configured to display an analysis result from the analysis unit,
wherein each of the light irradiation means and light detection means is arranged on/over the subject such that at least two types of SD distances are provided where the SD distance is defined as a distance between the irradiation point and detection point on/over the subject and that the SD distance is larger than approximately 10 mm, and
the analysis unit calculates, from a plurality of measurement signals measured in association as signals at certain measurement time with a combination of the light irradiation means and light detection means, at least one of a skin blood flow-derived signal and a brain blood flow-derived signal associated with the measurement time using SD distance dependency of the measurement signals.

2. The biophotonic measurement apparatus according to claim 1,
wherein a time waveform, an intensity distribution diagram, or the both of at least one of the skin blood flow-derived signals and the brain blood flow-derived signals is displayed during measurement.

3. The biophotonic measurement apparatus according to claim 1,
wherein a time waveform, an intensity distribution diagram, or the both of at least one of a skin contribution ratio and a brain contribution ratio is displayed during measurement.

4. The biophotonic measurement apparatus according to claim 1,
wherein an alert is output when the skin contribution ratio exceeds a preset threshold value or the brain contribution ratio is less than a preset threshold value.

5. The biophotonic measurement apparatus according to claim 1,
wherein the one or more light detection means detects signals of at least two types from the plurality of light irradiation means at different timings.

6. A biophotonic measurement method using a biophotonic measurement apparatus comprising one or more light irradiation means configured to irradiate a subject with light, one or more light detection means configured to detect, at a detection point on/over the subject, the light emitted to an irradiation point on/over the subject from the one or more light irradiation means and propagated within the subject, a control unit configured to control the one or more light irradiation means and the one or more light detection means, and an analysis unit configured to analyze a signal obtained by the one or more light detection means, the method comprising the step of:
arranging each of the light irradiation means and light detection means on/over the subject such that at least two types of SD distances are provided where the SD distance is defined as a distance between the irradiation point and detection point on/over the subject and that the SD distance is larger than approximately 10 mm; and
calculating, from a plurality of measurement signals measured in association as signals at certain measurement time with a combination of the light irradiation means and light detection means, a skin blood flow-derived signal and a brain blood flow-derived signal associated with the measurement time using SD distance dependency of the measurement signals.
